(19) United States
(12) Patent Application Publication
(10) Pub. No.: US 2026/0248533 A1
(43) Pub. Date: Aug. 27, 2026

(54) EXTERNAL FIXATION DEVICE FOR PREVENTING COMPARTMENT SYNDROME

(71) Applicant: KING SAUD UNIVERSITY, RIYADH (SA)

(72) Inventor: LEEN NASSER M ALRAJHI, RIYADH (SA)

(21) Appl. No.: 19/061,964

(22) Filed: Feb. 24, 2025

(51) Int. Cl. A61B 17/64; A61M 1/00

(52) U.S. Cl. CPC A61B 17/6425; A61M 1/91

(57) ABSTRACT

An external fixation device for preventing compartment syndrome includes a generally cylindrical body and a conical tip at a first end of the cylindrical body. The tip is configured for anchoring into the bone of the patient. The cylindrical body is configured for collecting fluid from surrounding tissue. A second end of the cylindrical body is free and can be connected to a negative pressure source to remove excess fluids received in the cylindrical body. Removal of the excess fluids from the surrounding tissue can reduce intra-compartmental pressure, providing a non-invasive solution for managing and preventing compartment syndrome during external fixation. The device combines external fixation with negative pressure wound therapy to prevent compartment syndrome.

### BACKGROUND

### 1. Field

The present disclosure relates to a device for preventing Compartment Syndrome and, particularly, to an external fixation device that is configured to apply Negative Pressure Wound Therapy (NPWT) for preventing Compartment Syndrome.

### 2. Description of the Related Art

Compartment Syndrome is a severe condition caused by increased pressure within a muscle compartment. Compartment syndrome is often due to trauma, fractures, or surgical interventions. The increased pressure within a muscle compartment restricts blood flow, leading to tissue damage and potential permanent disability. Traditional treatments, such as fasciotomy, are invasive and carry risks like infection and prolonged recovery.

There is an urgent need for an innovative and minimally invasive approach to decrease the intra-compartmental pressure associated with compartment syndrome, without relying on surgical intervention.

External fixation is a widely used orthopedic technique to stabilize fractures. However, the application of conventional external fixation measures can inadvertently increase the risk of developing compartment syndrome.

Thus, a device for preventing compartment syndrome solving the aforementioned problems is desired.

### SUMMARY

The present subject matter relates to a device for preventing compartment syndrome including a generally tubular or cylindrical body having a first end and an opposing second end, a peripheral wall extending between the first and second ends, and an inner body portion defined by a space between the peripheral wall and the first end. The first end can include a conical tip and the opposing second end of the cylindrical body can be free for detachably connecting with a negative pressure source. The tip can be a screw tip with threads extending along an outer surface thereof. The cylindrical body can include a mesh wall portion and a smooth wall portion. The mesh wall portion can include apertures defined therethrough to allow fluid to flow to an inner portion of the cylindrical body. The inner body portion of the cylindrical body can include a hollow buffer zone, a foam portion, and a plurality of upright support structures between the buffer zone and the foam portion. In an embodiment, the upright support structures can include a plurality of cylindrical tubes.

In an embodiment, a method for preventing compartment syndrome can include anchoring the device for preventing compartment syndrome into a bone of a patient to allow excess fluids from surrounding tissue to flow into the buffer zone of the device through the cylindrical tubes.

According to an embodiment, a device for preventing compartment syndrome in a patient can include a generally cylindrical body having a first end, an opposing second free end, a generally cylindrical peripheral wall extending between the first end and the second end, and an inner body portion defined by a space between the first end and the peripheral wall. The peripheral wall can include a perforated mesh wall portion and a smooth wall portion. A foam portion and a plurality of upright support structures can be disposed within the inner body portion. A plurality of upright support structures can surround the foam portion, the upright support structures being spaced from the peripheral wall, leaving an empty buffer zone between the upright support structures and the peripheral wall. The first end can include a conical tip including a screw tip with threads extending along an outer surface thereof. In an embodiment, the opposing second end of the cylindrical body is configured for detachably connecting with a negative pressure source.

These and other features of the present subject matter will become readily apparent upon further review of the following specification.

### BRIEF DESCRIPTION OF THE DRAWINGS

FIG. 1 is a perspective bottom view of the external fixation device for preventing compartment syndrome.

FIG. 2 is a perspective bottom view of the external fixation device for preventing compartment syndrome.

FIG. 3 is a bottom view of the external fixation device for preventing compartment syndrome

### DETAILED DESCRIPTION OF THE PREFERRED EMBODIMENTS

The following definitions are provided for the purpose of understanding the present subject matter and for construing the appended patent claims.

### Definitions

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

The term “optional” or “optionally” means that the subsequently described event or circumstance may or may not occur, and that the description includes instances where said event or circumstance occurs and instances in which it does not.

It will be understood by those skilled in the art with respect to any chemical group containing one or more substituents that such groups are not intended to introduce any substitution or substitution patterns that are sterically impractical and/or physically non-feasible.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

“Subject” as used herein refers to any animal classified as a mammal, including humans, domestic and farm animals, and zoo, sports, and pet companion animals such as household pets and other domesticated animals such as, but not limited to, cattle, sheep, ferrets, swine, horses, poultry, rabbits, goats, dogs, cats and the like.

“Patient” as used herein refers to a subject in need of treatment of a condition, disorder, or disease, such as cancer.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

According to an embodiment, the present subject matter relates to an external fixation device for preventing compartment syndrome. The external fixation device includes a generally cylindrical body and a conical tip at a first end of the cylindrical body. The tip is configured for anchoring into the bone of the patient. The cylindrical body is designed to collect fluid from the surrounding tissue with the assistance of negative pressure and, if necessary, the addition of hypertonic saline to enhance the removal of excess fluids from the tissue. This process helps reduce intra-compartmental pressure, providing a non-invasive solution for preventing compartment syndrome during external fixation. The device combines external fixation with negative pressure wound therapy (NPWT) to prevent compartment syndrome.

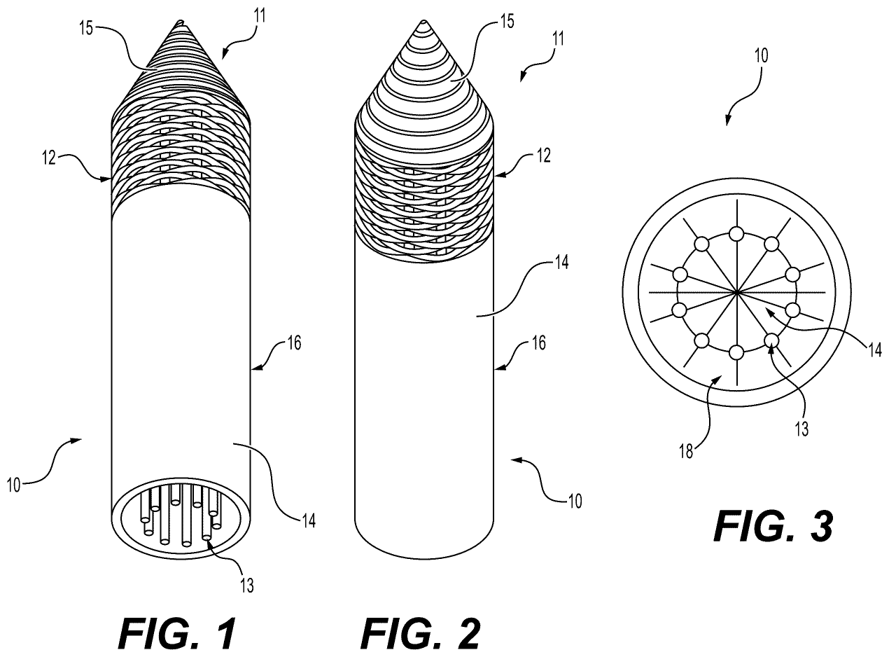

According to an embodiment, the present subject matter relates to a device for preventing compartment syndrome, designated 10 in the drawings. As shown in FIG. 1, the device 10 includes a generally tubular or cylindrical body 14. The cylindrical body 14 includes a first end, an opposing second free end, a generally cylindrical peripheral wall extending between the first end and the second end, and an inner body portion defined by a space between the first end and the peripheral wall. The first end includes a conical tip 11. The opposing second end of the cylindrical body can be free for detachably connecting with a negative pressure source. The tip 11 can be a screw tip with threads 15 extending along an outer surface thereof.

According to an embodiment, the cylindrical body 14 includes a mesh wall portion 12 and a smooth wall portion 16, as shown in FIG. 2. The mesh wall portion 12 includes apertures defined therethrough to allow excess fluid to flow to an inner portion 18 of the cylindrical body 16 once the device 10 is anchored into the bone. The smooth wall portion 16 provides a solid surface with no apertures.

According to an embodiment, the inner portion of the cylindrical body 16 includes a hollow buffer zone 18, a foam portion 17, and a plurality of upright support structures 13 between the buffer zone 18 and the foam portion 17, as shown in FIG. 3.

In an embodiment, the foam portion 17 can be disposed in a central portion of the cylindrical body 16, spaced from a peripheral wall of the cylindrical body 16. In an embodiment, the upright support structures 13 can be cylindrical tubes securely positioned within the cylindrical body 16 and configured to surround and secure the foam portion 17 once the foam portion 17 is disposed in the body 16. The upright support structures 13 can be configured to protect the foam portion 17 by preventing the foam portion 17 from tearing during positioning and by ensuring a gap is maintained between the foam portion 17 and the buffer zone 18. Thus, the foam portion 17 does not come into direct contact with the patient's body and maintains its structural integrity while in the cylindrical body 16.

In operation, the screw tip 11 can be securely anchored into the bone of a patient. Once secured to the bone, fluids from tissue surrounding the bone can flow into the buffer zone 18 of the device 10 through the mesh wall portion 12. The screw tip 11 ensures stability by securely anchoring the device 10 into the bone. This feature not only provides a firm foundation for the entire system but also stabilizes the fractured bone, aiding in effective fracture fixation. The buffer zone 18 regulates fluid movement, preventing direct contact between the surrounding tissues and the foam portion 17.

In an embodiment, the foam portion 17 can include a dressing foam typically used in negative pressure wound therapy (NPWT). For example, the foam portion 17 can include 3M™ V.A.C.® WHITEFOAM™ DRESSING

The buffer zone 18 can ensure that the foam portion 17 maintains its effectiveness without compromising tissue integrity or causing irritation. The upright support structures 13 protect the foam portion 17 and maintain the foam portion 17 within a central portion of the cylindrical body 16.

Once the device 10 is secured to the bone, the smooth wall portion 16 of the device 10 remains generally outside the body, enabling the connection of the second end of the device 10 to a negative pressure source. This connection facilitates the efficient removal of excess fluids, reducing intra-compartmental pressure and preventing complications such as compartment syndrome.

In an embodiment, the device for preventing compartment syndrome is configured to apply Negative Pressure Wound Therapy (NPWT) to a muscle compartment. In the past, (NPWT) has been successfully used to promote wound healing by applying continuous or intermittent sub-atmospheric pressure to the wound bed and removing excessive fluid. Despite its proven effectiveness in wound management, the potential of NPWT to prevent compartment syndrome has not previously been explored.

It is to be understood that the external fixation device for preventing compartment syndrome is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. An external fixation device for preventing compartment syndrome in a patient, comprising:
a generally cylindrical body having a first end, an opposing second free end, a generally cylindrical peripheral wall extending between the first end and the second end; and an inner body portion defined by a space between the peripheral wall and the first end, the peripheral wall including a perforated mesh wall portion and a smooth wall portion;
a foam portion within the inner body portion;
a plurality of upright support structures surrounding the foam portion, the upright support structures being spaced from the peripheral wall, leaving a hollow buffer zone between the upright support structures and the peripheral wall; and
a conical tip at the first end of the cylindrical body, the tip being a screw tip with threads extending along an outer surface thereof,
wherein the opposing second end of the cylindrical body is configured for detachably connecting with a negative pressure source.

2. (canceled)

3. The external fixation device of claim 1, wherein the plurality of upright support structures comprise a plurality of cylindrical tubes.

4. A method of preventing compartment syndrome in a patient, comprising:
anchoring the screw tip of the external fixation device of claim 1 into the bone of the patient;
collecting excess fluids from tissue surrounding bone in the foam portion;
connecting a negative pressure source to the second end of the cylindrical body; and
removing the excess fluids collected in the foam portion using the negative pressure source.

5. (canceled)

6. (canceled)

7. (canceled)
